(19) United States
(12) Patent Application Publication
(10) Pub. No.: US 2009/0292368 A1
(43) Pub. Date: Nov. 26, 2009

(54) PROSTHETIC LIMB CONNECTOR

(76) Inventors: John Plowman, Buderim (AU); Jacqueline Louise Plowman, Rosemount (AU)

Correspondence Address: TREGO, HINES & LADENHEIM, PLLC, 9300 HARRIS CORNERS PARKWAY, SUITE 210, CHARLOTTE, NC, 28269-3797, US

(21) Appl. No.: 12/355,109

(22) Filed: Jan. 16, 2009

Related U.S. Application Data
(60) Provisional application No. 61/021,336, filed on Jan. 16, 2008.

(51) Int. Cl. A61F 2/74

(52) U.S. Cl. 623/27

(57) ABSTRACT

A prosthetic limb connector for connecting a human prosthesis to an artificial base. The connector includes a connection body and a rotatable securing member to retain a portion of the prosthesis to the connection body.

### CROSS-REFERENCE TO RELATED APPLICATIONS

This application claims the benefit of Provisional Application 61/021,336, filed on Jan. 16, 2008.

### FIELD OF THE INVENTION

The present invention relates to improvements in devices designed to connect a human prosthesis to a base member attached to a human. More particularly, the present invention relates to improvements in devices designed to connect an amputee's stump socket, hereinafter termed “the socket,” of a prosthetic limb to the appropriate extremity, e.g. hand, foot, etc., which are examples of prostheses.

### BACKGROUND OF THE INVENTION

In conventional prosthetic limbs, the socket and the prosthesis are generally connected by a simple tube, known as the pylon, of aluminium or any suitably light and strong material. This is attached to the socket and to the prosthesis by means of bolts and/or screws. The removal and replacement of the prosthesis is time consuming, and requires the use of special tools. Moreover the refitting of the prosthesis involves a degree of re-adjustment. This has proved to be a burden, particularly for those sufferers who, for example, have alternative “feet” or “hands” to suit different activities. Even the simple operation of getting dressed or undressed in long trousers presents a considerable problem since the “foot” is rigidly attached at right angles to the “shin,” making it virtually impossible for the prosthesis to pass through the trouser leg.

At the end of the day it is often a relief to sit down and kick off one's shoes. The amputee can do this with the natural foot, but with the prosthetic foot it does not work. While it would be a relief to shed the prosthesis, conventional systems of attachment affords no ready means of doing this. There are many other situations where a quick release device would be desirable.

The present invention seeks to lessen these problems by providing a connecting device which allows the prosthesis to be attached to, and detached from the socket with relative quickness and ease, and with little or no special tools or re-adjustment.

### SUMMARY

The present invention in one preferred aspect provides for a connecting device adapted to connect human prostheses such as, but not limited to legs, arms, feet, hands and the like to an amputee with little or no need for special tools. In another preferred aspect, the present invention provides a method for connecting a prosthesis to an amputee. In a further preferred aspect, the invention provides for a connecting device and a set of prostheses useable with the connecting device.

### BRIEF DESCRIPTION OF THE FIGURES

FIG. 1 is a cross-sectional side view of a barrel member in accordance with a preferred embodiment of the present invention.

FIG. 2 is a cross-sectional side view of a connection body in accordance with a preferred embodiment of the present invention.

FIG. 3 is a cross-sectional side view of a nut in accordance with a preferred embodiment of the present invention.

FIG. 4 is a cross-sectional side view of a stem in accordance with a preferred embodiment of the present invention.

FIG. 5 is a cross-sectional side view of the barrel member, connection body, nut and stem of FIGS. 1 to 4 assembled together.

FIG. 6 is a cross-sectional top view of the stem of FIG. 4 taken along lines 6-6 of FIG. 5.

### DETAILED DESCRIPTION OF THE DRAWINGS

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

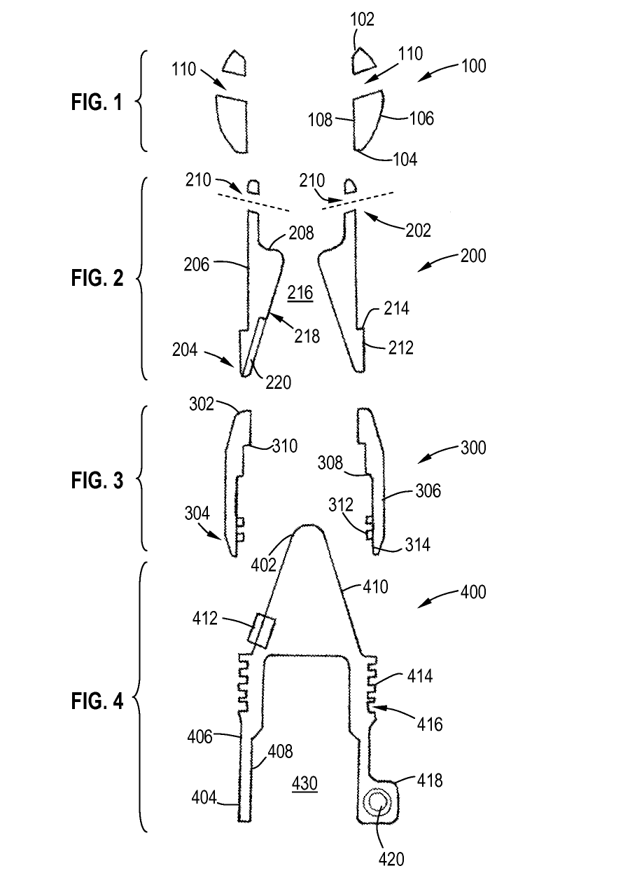

FIGS. 1 to 5 show a preferred embodiment of a limb connector 10 having a central longitudinal axis CLA, a barrel member 100 configured for attachment to a socket, a connection body 200 insertable into a portion of barrel member 100, a nut 300 and a stem 400 configured for placement on an end of a prosthesis for attachment with connection body 200 and nut 300. The preferred elements of limb connector 10 and their interrelationship are described below.

FIG. 1 shows barrel member 100 preferably configured for attachment on a socket. Barrel member 100 includes a first end 102, a second end 104 opposite first end 102, an exterior surface 106 and an interior surface 108. Exterior surface 106 is preferably convex along a vertical plane for engagement with a socket or other base member attached to a person. Interior surface 108 preferably includes parallel inner surfaces configured to receive a portion of connection body 200 therein. Preferably equally spaced around the central longitudinal axis are four angled holes 110, two of which are shown in FIG. 1. Holes 110 are preferably drilled and tapped to receive conventional grub screws. It will be understood and appreciated that the number and placement of holes and their angle relative to the central longitudinal axis may be varied without departing from the scope of the present invention.

FIG. 2 shows connection body 200 preferably adapted to longitudinally orientate a prosthesis relative to the central longitudinal axis of limb connector 10. As shown in FIG. 2, connection body 200 includes a first end 202, a second end 204, an exterior surface 206 and an interior surface 208. First end 202 preferably has an outer dimension compatible with the inner dimension of second end 104 of barrel member 100. First end 202 includes a plurality of holes 210 each having a mid-longitudinal axis preferably coaxial with a corresponding mid-longitudinal axis of one of holes 110 when first end 202 is inserted into barrel member 100. It will be appreciated that the number and placement of holes and their angle relative to the central longitudinal axis may be varied without departing from the scope of the present invention. Additionally, the number and positioning of screw holes associated with barrel member 100 may be different from the number and positions of screw holes associated with connection body 200. It will be appreciated that means other than screws may be used to secure connection body 200 to barrel member 100, for example, one or more bolts, a clamping means, or exterior threads on first end 202 to screw into second end 104 of barrel member 100. Examples of such means are described below with respect to other components of the limb connector and may be readily adapted for use between barrel member 100 and connection body 200.

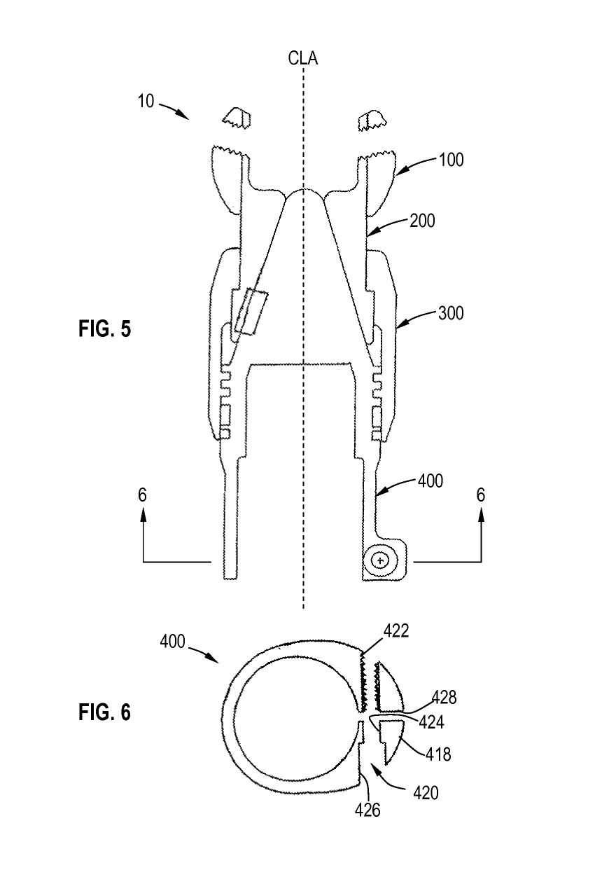

Exterior surface 206 preferably includes a shoulder 212 proximate second end 204 that forms a step 214 as shown in FIG. 2. Step 214 provides a surface upon which nut 300 may bear to bring connection body 200 and stem 400 firmly together, as shown in FIG. 5 and described in more detail below.

As shown in FIG. 2, interior surface 208 preferably forms a recess 216 extending between first and second ends 202, 204. A portion of recess 216 is preferably formed as a guide surface 218 in the shape of a female cone extending more than one-half the length of connection body 200 from second end 204. The shape of guide surface 218 is preferably formed to accommodate the shape of stem 400 as will be described in further detail below.

Guide surface 218 preferably includes at least one groove or keyway 220 extending from second end 204 configured to interact with a projection or key on stem 400 to facilitate and maintain rotational alignment of stem 400 with connection body 200.

FIG. 3 shows nut 300 preferably adapted to releaseably secure connection body 200 and stem 400 to one another. As shown in FIG. 3, nut 300 in a preferred embodiment is generally tubular in section. Nut 300 includes a first end 302, a second end 304, an exterior surface 306 and an interior surface 308. As shown in FIG. 3, exterior surface 306 includes parallel surface portions between two bevelled surface portions. The parallel surface portions may be knurled to provide grip in the hand operation of the mechanism.

FIG. 3 shows interior surface 308 including a stepped portion 310 proximate first end 302 to engage step 214 of connection body 200 (shown in FIG. 2). Interior surface 308 further includes a thread 312 for engagement with a corresponding thread on stem 400 (described below). Thread 312 preferably has a square profile as may be seen in FIG. 3. Thread 312 may have a sharp profile with a constant or varied pitch without departing from the scope of the present invention.

Second end 304 of nut 300 is preferably extended to form a lip 314 adapted to cover and compress at least a portion of an “O” ring against the exterior of stem 400.

FIG. 4 shows stem 400, which is preferably removeably attachable to a portion of a prosthesis. As shown in FIG. 4, stem 400 includes a first end 402, a second end 404, an exterior surface 406, and an interior surface 408. A portion of exterior surface 406 is preferably formed as a guide surface 410 in the shape of a male cone extending less than one-half the length of stem 400 from first end 402. The shape of guide surface 410 is preferably formed to accommodate the shape of the female cone in recess 216 of connection body 200.

Guide surface 410 preferably includes at least one projection or key 412 configured to interact with groove or keyway 220 of connection body 200 to facilitate and maintain rotational alignment of stem 400 with connection body 200. The number, position and shape of the projection may be varied without departing from the scope of the present invention.

Preferably the angle of guide surfaces 218 and 410 are substantially the same. It will be appreciated that the angle of guide surfaces 218 and 410 may differ from one another without departing from the scope of the present invention.

As shown in FIG. 4, stem 400 includes a thread 414 approximately midway along its length. Thread 414 preferably has a square profile as may be seen in FIG. 4. Thread 414 may have a sharp profile with a constant or varied pitch without departing from the scope of the present invention.

Exterior surface further preferably includes a groove 416 proximate the end of thread 414 closest to second end 404 to accommodate an O-ring (not shown). When the O-ring is engaged in groove 416, projecting lip 314 of nut 300 encloses the O-ring substantially within groove 416. The O-ring provides a degree of “drag,” similar to the action of a “nyloc” nut, inhibiting the accidental release of the nut in service.

Exterior surface 406 preferably includes at least one eccentrically enlarged portion 418 around the circumference of stem 400, shown in FIGS. 4 and 5. As shown in FIG. 6, enlarged portion 418 includes an opening 420 adapted to receive a clamping cross-bolt. Opening 420 preferably includes a thread 422, a non-threaded portion 424 and a countersunk portion 426 adapted to receive the head of the cross-bolt insertable into opening 420. Enlarged portion 418 further preferably includes a slit 428 parallel to the central longitudinal axis that permits the cylindrical portion of stem 400 to be divided longitudinally. Slit 428 may have a length greater than the height of enlarged portion 418 along the wall of stem 400. It will be appreciated that a clamping cross-bolt is preferred only and that other means for securing stem 400 to the prosthesis may be used without departing from the scope of the present invention.

Interior surface 408 of stem 400 preferably includes a recess 430 proximate second end 404. Recess 430 forms a parallel-sided cylinder of a dimension suitable to admit the introduction of a standard pylon tube, described above.

The shape of barrel member 100, connection body 200, nut 300 and stem 400 are preferably generally cylindrical in section, with the exception of the cross-section shown in FIG. 6, so one aspect of each component serves to convey its total profile. Barrel member 100, connection body 200, nut 300 and stem 400 are preferably machined, or formed by any other means such as molding, from aluminium or its alloys, or any other material having strength and low weight. Additional preferred materials include, but are not limited to titanium and its alloys, and polymeric materials.

Having described the components of limb connector 10, a preferred method of assembly thereof will now be described.

To fit the device to a prosthetic limb, barrel member 100 is attached to the standard pyramid block on the stump socket in the manner used to attach the pyramid adaptor in conventional prosthetic devices. This is achieved, for example, by means of the four grub screws referred to above. A removable stem 400 is attached to a pylon tube by inserting the tube into the split section proximate second end 404 of stem 400 (FIGS. 5 and 6). The prosthesis is rotated until the desired fore and aft, or rotational alignment of the foot or other artificial limb is achieved relative to key 412. The cross bolt is inserted to clamp the split section and fix the position of the prosthesis relative to key 412 of stem 400.

Once stem 400 has been attached to the pylon tube, the male cone at first end 402 of stem 400 is inserted into the female cone at second end 204 of connection body 200. Key 412 of stem 400 is guided along keyway or track 220 to maintain rotational alignment of the prosthesis relative to connection body 200. The key, having engaged the matching keyway in connection body 200, acts to ensure that this alignment will be maintained whenever the prosthesis is replaced in the preferred embodiment.

Once the male cone of stem 400 has been inserted into the female cone of connection body 200, the user rotates nut 300 to engage thread 312 with thread 414. Continued rotation of nut 300 will advance lip 314 of nut 300 against and over the O-ring (when present) positioned in groove 416 in stem 400 for friction-fit securement. To remove the prosthesis, rotation of nut 300 in the opposite direction preferably allows the user to remove or replace the prosthetic foot or other appendage quickly and easily, without tools or re-adjustment, and without removal of the socket.

It will be appreciated that the steps described above may be performed in a different order or certain steps omitted entirely without departing from the scope of the present invention. For example, stem 400 may be an integral part of a pylon tube or otherwise permanently attached to the pylon tube which eliminates the need for clamping stem 400 to the pylon tube.

The limb connection may be used with different types of prostheses, such as, but not limited to, feet, hands, legs and arms, or prostheses of the same type, but having a different shape or position.

The foregoing description is by way of example only, and may be varied considerably without departing from the scope of the invention. For example only, the male/female cone relationship of connection body 200 and stem 400 may be reversed, i.e., connection body 200 may include a male cone insertable into a female cone or funnel at the first end of stem 400. Guiding means other than cone-shaped surfaces may be used to guide and position connection body 200 and stem 400 relative to one another. The male cone at the first end of stem 400 may include one or more recesses that may optionally be in communication with recess 430 to reduce the over-all weight of the connection.

The positioning of the key and keyway may be reversed, i.e., connection body 200 may include one or more keys for interaction with one or more keyways in the guide surface of stem 400.

The thread proximate second end 304 of nut 300 may be external for engagement with an internal thread within stem 400 so that nut 300 may be at least partially rotated into stem 400.

Securing means other than nut 300 may be used to secure connection body 200 to stem 400. For example, the connector may be adapted for a resilient snap-fit engagement by omitting nut 300 entirely and incorporating a springlock in either or both of connection body 200 and stem 400. In such an embodiment, a portion of either connection body 200 or stem 400 would be inserted into the other until the spring lock engaged. The lock could be disengaged upon actuation by the user.

The securing means may include or form a clamping means. For example, the second end of connection body 200 may include a slit such as on stem 400. Once the stem is engaged with the connection body, the user may rotate a turn screw to compress the slit and secure the stem to the connection body. Another example of a clamping means would be the use of a partially resilient C-shape clip insertable in a groove formed through the sidewall of the second end of the connection body and into a matching groove in the sidewall of the stem. Insertion of the C-shape clip into the grooves of the connection body and the stem would clamp the stem to the connection body.

The present invention in a preferred form provides the advantages of being quick and easy to engage and disengage various types and shapes of prostheses. No tool is required in order to engage and disengage the connection. This provides the amputee more freedom and enhances the quality of life for the amputee.

It will of course be realised that the above has been given only by way of illustrative example of the invention and that all such modifications and variations thereto as would be apparent to persons skilled in the art are deemed to fall within the broad scope and ambit of the invention as herein set forth.

1. An apparatus for connecting a human prosthesis to an artificial base member attached to a human, said apparatus including:
a connection body having a first end adapted to attach to the base member, a second end opposite said first end, and a central longitudinal axis through said first and second ends, said second end including a guide surface adapted to slideably engage a portion of the prosthesis and axially orientate the prosthesis relative to the base member; and
a securing member adapted to rotate around the central longitudinal axis of said connection body to retain the portion of the prosthesis to said connection body.

2. The apparatus of claim 1, wherein said guide surface includes an interior adapted to receive the portion of the prosthesis therein.

3. The apparatus of claim 1, wherein said guide surface includes a projection adapted to be received into a portion of the prosthesis.

4. The apparatus of claim 1, wherein said guide surface is generally conical.

5. The apparatus of claim 1, further including the prosthesis, wherein the prosthesis includes a removable stem portion, the removable stem portion including the portion that slidably engages the guide surface.

6. The apparatus of claim 1, wherein said securing member includes a threaded portion.

7. The apparatus of claim 6, wherein said securing member includes an interior surface and an exterior surface, said threaded portion forming a part of said interior surface.

8. The apparatus of claim 6, wherein said securing member includes an interior surface and an exterior surface, said threaded portion forming a part of said exterior surface.

9. The apparatus of claim 1, wherein said securing member is a nut.

10. The apparatus of claim 1, wherein said guide surface includes a groove to facilitate axial orientation of the prosthesis relative to the base member.

11. A method of connecting a human prosthesis to an artificial base member attached to a human, said method including:
attaching a connection body to the base member, the connection body having a central longitudinal axis;
selecting a desired prosthesis to engage the connection body;
engaging the selected prosthesis with the connection body; and
rotating a securing member around the central longitudinal axis of the connection body to secure the selected prosthesis to the connection body.

12. The method of claim 11, wherein the step of rotating includes rotating the securing member more than one complete turn around the central longitudinal axis of the connection body.

13. The method of claim 11, wherein the step of engaging includes guiding a portion of the prosthesis along a track.
